(19) United States
(12) Patent Application Publication
(10) Pub. No.: US 2003/0054388 A1
(43) Pub. Date: Mar. 20, 2003

(54) Devices, methods and systems for high-resolution, high-throughput genetic analysis

(76) Inventors: Harold R. Garner, Coppell, TX (US); Kevin J. Luebke, Dallas, TX (US); Robert P. Balog, Dallas, TX (US)

Correspondence Address: Sanford E. Warren, Jr., Gardere Wynne Sewell LLP, Suite 3000, 1601 Elm Street, Dallas, TX, 75201-4761, US

(21) Appl. No.: 10/186,184

(22) Filed: Jun. 27, 2002

Related U.S. Application Data
(60) Provisional application No. 60/301,317, filed on Jun. 27, 2001.

(51) Int. Cl. C12Q 1/68; G06F 19/00; G01N 33/48; G01N 33/50

(52) U.S. Cl. 435/6; 702/20

(57) ABSTRACT

An apparatus and method for comparative genomic mapping of a sample and a reference is disclosed that includes an array of oligonucleotides having a surface derived from a selected portion of a genome. A reader capable of producing a signal disposed to detect events that occur at the surface of the array of oligonucleotides and a computer attached to the reader, wherein the computer processes a signal from the reader to determine the relative intensity produced at the array surface and capable of comparing the intensity of the signal from the array to provide map with a resolution of about 20, 30, 50, 100, 200, 500, 1,000, 2,000, 5,000, 20,000, 50,000, 100,000, 200,000, 500,000, 1,000,000, 2,000,000 basepairs.

This application claims priority to U.S. Provisional Patent Application Serial No. 60/301,317 filed Jun. 27, 2001.

The U.S. Government may own certain rights in this invention pursuant to the terms of the National Cancer Institute grant CA81656-01.

### TECHNICAL FIELD OF THE INVENTION

The present invention relates in general to the field of automation and high throughput analysis for biological sample analysis, and more particularly, to devices and methods for the high-resolution analysis of genetic amplifications and deletion.

### BACKGROUND OF THE INVENTION

Without limiting the scope of the invention, its background is described in connection with techniques of comparative genomic analysis for the detection of somatic diseases, as an example. The analysis of biologically relevant samples has long been accomplished using techniques that detect the presence of a marker or markers from known and unknown samples. To detect the presence of these markers, techniques such as, e.g., radiolabelling, fluorescence or enzymatic labeling, have been used to detect the presence or absence of binding between a component of the sample and a substrate or matrix on which the appropriate binding group or ligand has been immobilized. These detection techniques have been expanded to the analysis of genetic mutations and deletion at both the macroscopic and microscopic level.

Genetic deletions and copy number increases are associated with many diseases (Pinkel, et al. (1998) Nat. Genet. 20, 207-211). For example, the transcriptional effects of deletion of tumor suppressor genes or their regulatory elements are associated with cancer. Copy number increases of oncogenes similarly contribute to tumorigenesis. Furthermore, a number of developmental abnormalities result from gain or loss of a chromosome or a region of a chromosome. Thus, there is a need for a means to detecting and mapping abnormalities of copy number. Such information enables association of disease phenotypes with particular genetic aberrations, which is valuable to understanding disease processes and precise diagnosis of diseases.

Traditionally, chromosomal aberrations have been detected and mapped by visual inspection of a metaphase chromosomal spread. This method, termed karyotyping, is limited, however, by the difficulty of producing many high-quality metaphase spreads and by the difficulty of visually analyzing complex chromosomal changes (Teyssier, et al. (1989) Cancer Genet. Cytogenet. 37, 103). Molecular genetic techniques, such as restriction fragment length polymorphism (RFLP) analysis, can also reveal chromosomal abnormalities such as allelic loss, but such methods must focus on one or a few chromosomal loci at a time (Fearon, et al. (1990) Cell 61, 759).

Yet another technique is Comparative Genomic Hybridization (CGH), which permits the creation of a genomic map of DNA sequence copy number as a function of location on the chromosome (Kallioniemi, et al. (1992) Science 258, 818-821). Thus, CGH provides a more quantitative description of chromosomal structure than karyotyping and provides a more comprehensive view of chromosomal structure than molecular genetic techniques like RFLP analysis. In its earliest form, CGH was performed by allowing two DNA samples, a test and a normal reference sample, to hybridize to a metaphase spread of normal chromosomal DNA. The two samples are labeled with fluorescent dyes of different colors so that they can be detected independently. The relative amounts of test and reference DNA bound at any position on the chromosome depend upon the relative abundances of the sequence at that position in the two DNA samples. The relative amounts of test and reference DNA hybridized are determined by quantitating the amount of signal from each fluorescent dye.

One limitation of CGH is that it requires a metaphase chromosomal spread, which is difficult to implement in a high-throughput or automated assay. Another type of CGH circumvents the requirement for a metaphase chromosomal spread by hybridizing the sample to arrays of large genomic DNA clones (e.g., BACs) or even smaller DNA, such as cDNAs (Pollack et al. (1999) Nat. Genet. 23, 41-46). Hybridization of test samples to arrays of cDNAs offers the additional advantage that DNA copy number and gene expression level may be characterized in parallel, with the same sample and array.

Nevertheless, current CGH analysis using array techniques suffers from certain drawbacks. For example, when large genomic DNA clones are used on the array (e.g., BACs), the resolution at which genetic aberrations are mapped is limited by the size of the clones and the extent to which the genetic aberrations and the clones on the array overlap, typically 20 kbp. Mapping with cDNA arrays, on the other hand, limits the CGH analysis to coding regions of the genome and does not address directly alterations in regulatory or other sequence.

### SUMMARY OF THE INVENTION

It has been found, however, that the present methods do not permit flexibility in the resolution of the genetic analysis. Furthermore, present methods require, either, the isolation and handling of very large fragments (e.g., BACs) or are limited to coding and adjacent transcribed sequence. Yet another significant problem of current methods is the lack of detailed mapping necessary for the medical diagnosis of complex genetic diseases.

The present invention provides a high-throughput method for high resolution mapping of genetic copy number variations between samples of DNA. The method uses an array of probes that bind specifically to short sequences (about 20 nucleotides) in the genome. These probes can be placed at virtually any desired spacing along the genome to define the resolution of the mapping. The amount of hybridized DNA is related to the abundance of the sequence of interest. A device is provided comprising an array of probes, and principles for its design and fabrication are included.

In one embodiment of the present invention, genomic DNA from test and reference samples is allowed to hybridize to an array of oligonucleotides, 16-100 nucleotides in length, immobilized on a surface. The oligonucleotides are chosen to probe a region of interest that might include the entire genome. Binding of test and reference samples to each probe is quantitated after washing unbound DNA away. The relative amounts of hybridized test and reference DNA to each probe indicated the relative abundances of the sequences complementary to that probe in the test and reference samples.

More particularly, the present invention is an apparatus and method for mapping a sample at high resolution that includes the steps of, selecting a portion of a genome for genetic analysis; providing an array of oligonucleotides specific to the portion of the genome selected for genetic analysis; and conducting a comparative genomic hybridization analysis, whereby the resolution of the analysis is between about 20 and 2,000,000 basepairs.

Another embodiment of the present invention is a method for high-throughput high resolution mapping of a sample that includes the steps of; selecting a portion of a genome for genetic analysis; providing an array of oligonucleotides specific to the portion of the genome selected for genetic analysis; hybridizing a sample nucleic acid and a reference nucleic acids to the array; detecting the binding of DNA to specific portions of the genome; and conducting a comparative hybridization analysis between the sample and the reference nucleic acids.

Yet another embodiment of the present invention is an apparatus for comparative genomic mapping of a sample and a reference, including: an array of oligonucleotides having a surface derived from a selected portion of a genome, a reader capable of producing a signal disposed to detect events that occur at the surface of the array of oligonucleotides and a computer attached to the reader. The computer processes a signal from the reader to determine the relative intensity produced at the array surface and capable of comparing the intensity of the signal from the array to provide map with a resolution between 20 basepairs and 2,000,000 basepairs. In one alternative embodiment the limit of resolution of the genetic map is between about 20, 30, 50, 100, 200, 1,000, 2000, 5,000, 20,000, 50,000, 100,000, 200,000, 500,000, 1,000,000, and 2,000,000 base pairs; about 200 bases and 100 kilobases; about 5 and 50 kilobases and even about 10 and 15 kilobases.

Samples for use with the present invention include, e.g., histological or blood samples, and may even include partially isolated or even in situ DNA, e.g., genomic DNA. The array may be on, e.g., a slide or wafer.

The present invention also includes a system for oligonucleotide design and the oligonucleotides designed therefrom. The system includes the steps of inputting a portion of a genome sequence file in 5′ to 3′ direction; generating a complementary sequence in the 3′ to 5′ direction to create a parent probe list; and creating a final probe list by: filtering probe sequences from the final probe list that are not suitable for hybridization analysis from the parent probe list; and outputting a file containing all the probes for each position in the reference sequence.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a illustrative drawing showing the signal detected from an array wherein an increasing number of labeled probe is attaching to an array at a specific location;

FIG. 2 is a graph showing the comparative genomic hybridization signal detected on an array using the present invention; and

FIG. 3 is an image generated by the scanner software were converted to 16-bit TIFF format and analyzed using the DOC_U_MENTOR code.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used throughout the present specification the following abbreviations are used: TF, transcription factor; ORF, open reading frame; kb, kilobase (pairs); UTR, untranslated region; kD, kilodalton; PCR, polymerase chain reaction; RT, reverse transcriptase.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

The inhibition of hybridization of the completely complementary sequence to the target sequence may also be examined using a hybridization assay involving a solid support (e.g., an array). The absence of non-specific binding may be tested by the use of a second target that lacks even a partial degree of complementarity (e.g., less than about 30% identity). In the absence of non-specific binding, the probe will not hybridize to the second non-complementary target and the original interaction will be found to be selective.

Low stringency conditions are generally conditions equivalent to binding or hybridization at 42 degrees Centigrade in a solution consisting of 5×SSPE (43.8 g/l NaCl, 6.9 g/l NaH2PO4-H₂O and 1.85 g/l EDTA, pH 7.4), 0.1% SDS, 5× Denhardt's reagent (50× Denhardt's contains per 500 ml: 5 g Ficoll (Type 400, Pharmacia), 5 g BSA (Fraction V; Sigma) and 100 micrograms/ml denatured salmon sperm DNA); followed by washing in a solution comprising 5×SSPE, 0.1% SDS at 42 degrees Centigrade when a probe of about 500 nucleotides in length is employed.

The art knows that numerous equivalent conditions may be employed to achieve low stringency conditions. Factors that affect the level of stringency include: the length and nature (DNA, RNA, base composition) of the probe and nature of the target (DNA, RNA, base composition, present in solution or immobilized, etc.) and the concentration of the salts and other components (e.g., formamide, dextran sulfate, polyethylene glycol). Likewise, the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, inclusion of formamide, etc.).

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

The term “portion of a genome for genetic analysis” or “chromosome-specific” is herein defined to encompass the terms “target specific” and “region specific”, that is, when the staining composition is directed to one chromosome or portion of a genome, it is chromosome-specific, but it is also chromosome-specific when it is directed, for example, to multiple regions on multiple chromosomes, or to a region of only one chromosome, or to regions across the entire genome. Likewise, “locus specific” or “loci specific” is defined as locations on one or more chromosomes for a particular gene or allele. Sequence from regions of one or more chromosomes are sources for probes for that region or those regions of the genome. The probes produced from such source material are region-specific probes but are also encompassed within the broader phrase “portion of a genome” probes. The term “target specific” is interchangeably used herein with the term “chromosome-specific” and “portion of a genome”.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library”. Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected sequence.

“Staining reagent” refers to the overall hybridization pattern of the nucleic acid sequences that comprise the reagent. A staining reagent that is specific for a portion of a genome provides a contrast between the target and non-target chromosomal material.

A “probe” as defined herein may be one or more molecules that can hybridize to a nucleic acid target sequence and that can be detected (e.g., nucleic acid fragments or other oligomers that bind nucleic acids). Examples of possible probe molecules include, but are not limited to, DNA, RNA, peptides, minor groove-binding polyamides, peptide nucleic acids (PNA), locked nucleic acids (LNA), and 2′-O-methyl nucleic acids. The probe is labeled so that its binding to the target can be assayed, visualized or detected. In essence the probe is designed to bind a target, also referred to as an analyte, so that the combination of probe and analyte may be assayed, visualized or detected. The probe may be produced from some source of nucleic acid sequences, for example, a collection of clones or a collection of polymerase chain reaction (PCR) products or the product of nick translation or other methods for adding a detectable marker to a nucleic acid binding moiety. For nucleic acids, repetitive sequences are removed or blocked with unlabeled nucleic acid with complementary sequence, so that hybridization with the resulting probe produces staining of sufficient contrast on the target. The word probe may be used herein to refer not only to a molecule that detects a nucleic acid, but also to the detectable nucleic acid in the form in which it is applied to, e.g., the surface of an array. What “probe” refers to specifically should be clear to those of skill in the art from the context in which the word is used.

The term “labeled” as used herein indicates that there is some method to visualize or detect the bound probe, whether or not the probe directly carries some modified constituent. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities.

Multiple methods of probe detection may be used with the present invention, e.g., the binding patterns of different components of the probe may be distinguished—for example, by color or differences in wavelength emitted from a labeled probe.

A number of different aberrations may be detected with any desired staining pattern on the portions of the genome detected with one or more colors (a multi-color staining pattern) and/or other indicator methods.

The phrase “high complexity” is defined herein to mean that the probe, thereby modified contains on the order of 50,000 (50 kb) or greater, up to many millions or several billions, of bases of nucleic acid sequences that are not repeated in the probe. For example, representative high complexity nucleic acid probes of this invention can have a complexity greater than 50 kb, greater than 100,000 bases (100 kb), greater than 200,000 (200 kb), greater than 500,000 bases (500 kb), greater than one million bases (1 Mb), greater than 2 Mb, greater than 10 Mb, greater than 100 Mb, greater than 500 Mb, greater than 1 billion bases and still further greater than several billion bases.

The term “complexity” is defined herein according to the standard for nucleic acid complexity as established by Britten et al., Methods of Enzymol., 29:363 (1974). See also Cantor and Schimmel, Biophysical Chemistry: Part III: The Behavior of Biological Macromolecules, at 1228-1230 (Freeman and Co. 1980) for further explanation and exemplification of nucleic acid complexity.

The complexity for a final probe list and array will depend on the application for which it is designed (e.g., location on the genome, complexity of the sequence, etc.) and the mapping resolution that is sought. In general, the larger the target area, the more complex the probe list. The term “complexity” therefore refers to the complexity of the total probe list no matter how many visually distinct loci are to be detected, that is, regardless of the distribution of the target sites over the genome.

The hybridization techniques it is possible to obtain a reliable, easily detectable signal with a probe of about 0.02 kb to about 2,000 kb targeted to a compact point in the genome. For example, a complexity in the range of approximately 0.02 kb permits hybridization to both sides of a tumor-specific translocation. The portion of the probe targeted to one side of the breakpoint may be labeled differently from that targeted to the other side of the breakpoint so that the two sides can be differentiated with different color. Proportionately increasing the complexity of the probe permits analysis of multiple compact regions of the genome simultaneously. Changes in the copy number of a gene, or even specific mutations and deletions at the basepair level may be detected using the present invention with a series of probe-based, color coded (for example), reference points along each chromosome or significant regions thereof. The detection may even include the level of expression of a gene by comparing the level of mRNA to the genomic number of a locus or loci.

Uniform staining of an extended contiguous region of a genome, for example, a whole chromosome may requires a probe complexity proportional to but substantially less than, the complexity of the target region if using a single array, or if using multiple arrays then the level of resolution can be increased. The total nucleic acid concentration of the probe may be varied to provide the best level of detection (e.g., signal to noise ratio). A decrease in concentration of a portion of a sequence in the probe may be compensated for by increasing the number of target sites using, e.g., overlapping probes. In fact, when using double-stranded probes, a relatively low concentration of each portion of sequence may inhibit reassociation before a portion of the sequence can find a binding site in the target.

A locus at any point in the genome may either “single-copy” or “repetitive”, however, for use with the present invention the oligonucleotide on the array and its sequence needs to be long enough so that a complementary probe sequence can form a stable hybrid with the target sequence under the hybridization conditions being used. Such a length is typically in the range of tens to hundreds of nucleotides.

A “single-copy sequence” is where one copy of the target nucleic acid sequence is present in the haploid genome. “Single-copy sequences” are also known in the art as “unique sequences”. A “repetitive sequence” is where more than one copy of the same target nucleic acid sequence in the genome. Each copy of a repetitive sequence need not be identical to all the others. An important feature is that the sequence should be sufficiently similar to the other members of the family of repetitive sequences such that under the hybridization conditions being used, the same fragment of probe nucleic acid can form stable hybrids for each copy. A “shared repetitive sequence” is a sequence with some copies in the target region of the genome, and some elsewhere.

The required contrast (e.g., signal to noise) for detection will depend on the application for which the probe is designed and even the portion of the genome that is the target of the analysis. When visualizing chromosomes and nuclei, etc., microscopically, a contrast ratio of two or greater is often sufficient for identifying whole chromosomes. When quantifying the amount of target region present on an array by fluorescence intensity measurements using a slide reader or quantitative microscopy.

For manufacture of the DNA binding oligomer array on glass may be accomplished using techniques known in the art for covalent attachment of oligomers to silica or other substrate, e.g., covalent crosslinkers may be added to the glass that modify the surface in a manner that permits binding of the oligomer to the glass via the crosslinker. Another example may be a mixture of proteins that cause reduced background signals to the target nucleic acids, the application of the mixture to the matrix and a subsequent fixing. Fixation to the protein of the oligomer may be via, e.g., methanol/ice vinegar or formaldehyde. The substrate for attachment of the DNA binding oligomer may be glass or other hard materials. One such example may be microplates with pre-formed cavities.

The measurement after binding on the array may be performed in a batch for each reaction, which is particularly amenable to automation, e.g., using a slide reader. The signals of the sample and reference nucleic acids are determined in accordance with the signal characteristic. For example, sample and reference nucleic acids may be marked by different fluorochromes. Both fluorochromes may be excited concurrently or in series, and measured separately or they may be simultaneously excited and detected separately.

Another option for labeling the nucleic acids is by adding haptenes (for example, biotin or digoxigenin) or directly using fluorochromium is done by using standard molecular-genetic procedures (Nick Translation, PCR and/or Random Priming)

The Comparative Nucleic Acid Hybridization is performed as follows. The hybridized sample sequences are detected by binding the sample and reference nucleic acids to the array and generating quantitatively measurable signals that are sufficiently distinguishable from “background” signals of the array. For this purpose, fluorescent properties are useful. With fluorochromium-marked nucleic acids, e.g., the sample sequences are directly detected after the washing steps.

Fluorochromes or dyes for use with the present invention will depend on wavelength and coupling structure compatibility. By means of example, Fluorescein-5-EX, 5-SFX, Rhodamine Green-X, Bodipy FL-X, Cy2-OSu, Fluor X, 5(6)TAMRA-X, Bodipy TMR-X, Rhodamine Red-X, Texas Red-X, Bodipy TR-X, Cy3-OSu, Cy3.5-OSu, Cy5-Osu and/or Cy5.5-OSu, may be used if desired.

Fluorescence detection reactions by haptene-marked nucleic acid samples is performed in accordance with standard procedures. Other detection methods may be used which will provide quantifiable signals, such as chemical luminescence, phosphorescence and radioactivity in order to directly or indirectly determine the presence of nucleic acids. Different detection methods for the test and reference nucleic acids may also be combined in a single study.

In an automated setting, the present invention may be used to detect hybridization signals using, e.g., a charge coupled display camera (CCD camera). The data captured by the camera, namely, location (x, y) and intensity is determined for the one or more wavelengths and the fluorescence of the sample nucleic acid and/or the reference nucleic acid is calculated by a computer using methods known in the art of karyotyping and comparative genomic analysis.

In samples having a genomic duplication or deletion of a chromosome, of a chromosome section or of a gene may be determined by the identification of a systematic increase or decrease in the signal detected for the respective nucleic acids. The fluorescence emitted for the remainder of the sample in which detection has not occurred should remain within the control range. The hybridization signal resulting from the sample genome is compared with that resulting from, e.g., the normal reference DNA. The array should be relatively insensitive with regard to variations in the amount of target nucleic acids in the various locations on which the specific oligonucleotides have bee attached to the array. Variations in the mixing ratio of the sample nucleic acids and the reference nucleic acids may occur in different studies.

The equipment for the quantitative determination and mapping using hybridization signals, include measuring linear differences between signal intensities, e.g., over a wide range will depend on the level of resolution required for mapping.

For the detection of fluorescence signals various instrument configurations may be used such as: fluorescence microscopes that include a (cooled) CCD (Charged Coupled Device) camera or scanners, where fluorescence scanning may be performed by way of an electronically controlled light source (e.g., a laser beam) and detection occurs by way of a sensitive photo-multiplier. Depending on the type of detection signals also other methods such as densitometry (see for example, phosphorous imaging) are suitable. All measurement data may be digitally recorded and stored and the relative signal intensities of sample and reference nucleic acids may then be calculated using suitable software.

The present invention may be used in the areas of clinical genetics, tumor diagnostic, clinical pathology, the analysis of animal models for genetic diseases including tumors and in breeding research.

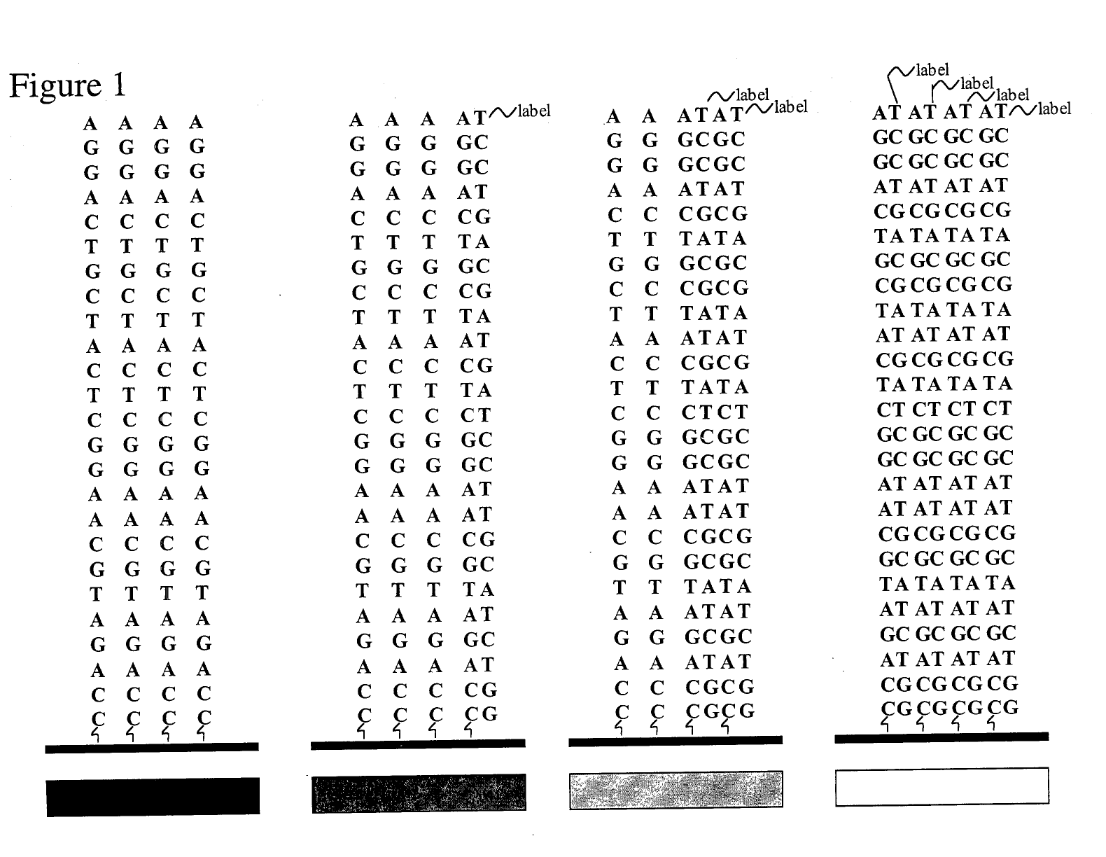

High-resolution mapping of genomic sequence copy number can be accomplished in an automated and high-throughput way by allowing the sample of DNA to bind to an array of sequence-specific probes that bind specifically to short sequences of the genome. The amount of hybridized DNA is related to the abundance of the DNA (FIG. 1) in the sample and can be detected and quantitated in a variety of ways. An array of probes that bind to short sequences affords the highest potential resolution to the mapping. The probes must, however, bind specifically to sequences that are sufficiently long for their abundances to be meaningful. Because a sequence of 16 nucleotides is expected to be unique in a random sequence of 3 billion nucleotides, the abundances of sequences 16 base pairs and longer are most informative about chromosomal structure at the level of functional units.

A number of formats for probe arrays have been used, but one that is particularly relevant to the present invention is an array of oligodeoxyribonucleotides immobilized on a glass surface. A “feature” of the resulting array is defined as a region of the surface in which a single probe sequence predominates. Fabrication of surface-bound oligonucleotide arrays can be accomplished by a variety of methods known to those with skill in the art.

A fabrication method that is particularly appropriate for the present invention makes use of light directed chemistry to synthesize the oligonucleotides directly on the surface (Pease, et al. (1994) Proc. Natl. Acad. Sci. USA 91, 5022-5026). The regions of the surface that are illuminated during pre-determined chemical steps of the synthesis determine the sequence synthesized in each feature. Defined regions can be illuminated discretely by, for example, shining light through a physical mask that blocks light from particular regions or by directing light to particular regions with a digital micromirror array (Jaklevic, et al. (1999) Annu Rev. Biomed. Eng. 1, 649-678, and Singh-Gasson, et al., (1999) Nat. Biotech 17, 974-978). These light-directed approaches are preferred for the present invention, because they currently enable the largest numbers of features per unit area of array surface. Thus, the highest resolution mapping and the greatest coverage of the genome are accomplished with the very high feature numbers accessible with light-directed methods. However, other methods of array fabrication are amenable to the present invention, including but not limited to delivering the reagents of DNA synthesis to specific regions of the surface and depositing on the surface oligonucleotides that have been pre-synthesized.

Digital Optical Chemistry. The array of the present invention may be created using an apparatus that catalyzes a reaction on a substrate using light activated chemistry. In this system, a light source is directed toward a micromirror positioned to redirect light from the light source toward a substrate. A computer is connected to, and controls, the micromirror and a substrate holder, such as a reaction chamber, that is placed in the path of light redirected by the micromirror, wherein light that is redirected by the micromirror catalyzes a chemical reaction proximate the substrate. By proximate it is meant that the light catalyzed reaction can occur on or about the surface of the substrate. A light source may be a lamp or laser, such as a UV light. In an alternative embodiment the light source may be, e.g., a xenon lamp, or a mercury lamp, or a laser or a combination thereof. The light produced by the light source can also be visible light. One advantage of catalyzing chemical reactions using UV light is that it provides photons having the required high energy for the reaction and it can be directed to create smaller and smaller features, depending on the size of the array. UV light is also advantageous due to its wavelength providing high resolution. Lenses are positioned between the light source and the micromirror, which can be a micromirror array, or between the micromirror and the substrate. An example of such a lens is a diffusion lens.

The light catalyzed synthesis or reaction can be, e.g., the addition a nucleotide base to the substrate or to a base or polynucleotide chain attached to the substrate. Likewise, the light redirected by the micromirror can catalyze a chemical reaction, e.g., an amino acid addition reaction or the addition, removal or crosslinking of organic or inorganic molecules or compounds, small or large. For example, during the addition of a nucleic or an amino acid residue, the light can deprotect protecting groups of, e.g., phosphoamidite containing compounds. Light can also be responsible for the crosslinking or mono-, bi-, or multi-functional binding groups or compounds to attach molecules such as, fluorochromes, antibodies, carbohydrates, lectins, lipids, and the like, to the substrate surface or to molecules previously or concurrently attached to the substrate.

A method of patterning on a substrate may include the steps of, generating a light beam, illuminating a micromirror with the light beam, redirecting the light beam with the micromirror onto a substrate and catalyzing a light sensitive reaction proximate to the surface of the substrate using the redirected light beam in a predetermined pattern. By using the method of the present invention as a series of cycles, a number of layers can be built on the substrate or strings of molecules can be built having a large diversity. The method of the present invention can further comprising the step of controlling, using a computer, the micromirror, which can be a light mirror array such as, e.g., a Texas Instruments Digital Light Processor, a waveguide, and LCD array or even a liquid crystal display. The illuminating light beam may be a UV, or other light source that is capable of catalyzing a chemical reaction, such as the formation of a positive or negative photoresist. The present method can also be used for the in situ addition or removal of organic or inorganic molecules or compounds, as will be known to those of skill in the art of photochemistry. In addition, mask based technology may be used to produce the arrays for use with the present invention.

The present invention can be used, e.g., in “stepper” fashion, wherein the micromirror is directed at a portion of the substrate, that portion of the substrate exposed to light from the micromirror, and then stepped on to a different portion. The new portion of the substrate exposed can be, e.g., overlapping or adjacent to the first portion.

Following are the specifications and characteristics for one embodiment of the micromirror imager system for use with the present invention:

Control computer—PC with VGA monitor, or Macintosh

Software—Image created using PowerPoint, custom Software or CAD software

Digital Light Processor—TI DLP with 640×480 resolution

Number of pixels—640×480=307,200

Mirror material—Aluminum

Mirror reflectivity—88%, Verified using monochromater/PM tube for visible and UV wavelengths

Mirror size—16 microns×16 microns in a 20 micron×20 micron space

Synthesis spot size—1:1 with mirror size

Mirror switching speed—2 ms

Light source—100 W mercury burner with peak at 365 nm

Light brightness—170,000 cd/cm2=250 W/(cm2*st)

Luminous Flux—2,200 lumens

Reaction chamber—custom from teflon, delrin and aluminum

Reagent delivery—syringe injectors into header

Sample configuration—coated microscope slides

Microscope slide transparency—5%@280, 40%@300, 75%@320, 87%@340, 88%@360, 89%@400, measured using spectrophotometer

Exposure time—3 minutes per coupling reaction.

The apparatus and method of the present invention has been used to: 1) show that the mirror array can project UV light (UV light cannot be passed through conventional liquid crystal displays) at sufficient intensity to conduct photochemistry, 2) demonstrate that images at the focal plane can be created, and 3) demonstrate the use of the apparatus and method photodeprotection chemistry to make an patterned substrate.

The optics for use with a digital optical chemistry apparatus to make the array may be designed to maintain the system focus while substantially increasing the contrast ratio. A high contrast ratio is critical to obtain high quality differential synthesis that is a function of UV intensity and exposure time. The critical component to obtaining higher contrast ratios is the TIR (Total Internal Reflectance) prism, which escorts the LV light from the source onto the substrate and then out to the focusing optics. These optics may be customized for a particular DLP with, e.g., UV transparent glass (BK5, SF5 or K5). The use of a TIR prism is not necessary, as the apparatus and method of the present invention has been used with direct projection via a mirror set having 20 degrees off-axis of the micromirror to match the cant angle of the individual mirrors and lenses. The TIR prism and lens 22, e.g., Acromat doublets or triplets, can be made from UV transparent fused silica (many types are available for 365 nm, near UV).

A high power UV source can be used, e.g., a power source of up to 1 kW can be used before reaching a damage threshold for the micromirror. Also, an automated liquid handling system can be constructed, fashioned from that used in the MerMade Oligo Synthesizer (UTSW Medical Center, U.S.A.) or other commercially available synthesizers (Beckman Instruments or Applied Biosciences, Inc., U.S.A.). Reagents can be kept in, e.g., Argon pressurized bottles and dispensed through teflon coated valves under computer control. A National Instruments digital I/O board can be installed in the Macintosh control computer, followed by a solid state relay system that provides the level of current necessary to run the valves, which can be e.g., microvalves. Under computer control, the valves can be opened between 100 msec and 1 sec, depending on the amount of reagent to be dispensed. The control software for the valves may be Labview or other custom codes written in e.g., C or other computer languages. Pressurized bottles and valves can be provided for each reagent.

The reagents will be delivered to, e.g., the slide holder on which the light is projected. The slide holder may be fabricated from Teflon, with slides sealed using o-rings. Two slides can be sandwiched with the reagents pumped between them. The two-slide sandwich arrangement allows for the manufacture of two slides, concurrently, and minimizes the scattered light for excess light is projected through sandwich. The entire sandwich can be clamped together and Luer lock fittings will be used to attach the liquids.

The substrate, slide(s) or other surface for use with the invention may be made of different materials, such as silicon, glass or quartz. The slides may also have patterns on the surface that are useful for increasing the attachment of compounds. Additionally, the slide surface may be formed or modified to increase the surface area and consequently the amount of material that is formed, deposited or catalyzed on the slide surface. One example of a modified surface area slide for use with the invention is a microchannel slide that has a number of grooves or channels throughout the slide that increase the surface area of the slide. Another examples of a surface enhancing feature include: dimples, holes, scratches and fibrous deposits or mesh.

It should be noted, that once produced the digital optical chemistry (DOC) slide arrays when used may have a modified hybridization protocol. Possible adjustments will include temperature, time, sample concentration, buffer and wash conditions. These can be resolved depending on the background and signal to noise ratio encountered with an initial DOC slide. Once the DOC has been manufactured, however, the same slide can be stripped and reused for the next hybridization cycle with new conditions. Alternatively, the image acquisition can be recalibrated to take into account increased background signal to improve the signal to noise ratio through adjustments, manual or automatic, to the data acquisition software. In fact, sample data can be taken from the positive and negative controls on the DOC slide by, e.g., placing the positive and negative controls in the first line of samples to be scanned, and adjusting the calibration for the entire DOC chip before any more data acquisition continues.

Photoprotection chemistries for improved coupling yield. The critical step with light-directed synthesis of DNA arrays on glass supports is the rate of photolytic release of the 5′-protecting group that is related to the reaction quantum efficiency. For the 2-nitrobenzylic compounds to be used in proposed research, the 365 nm emission of the Hg lamp is almost exclusively responsible for photochemistry due to its chromophore absorbance (λₘₐₓ=345 nm, ε=5×10³ M⁻¹ cm⁻¹). The photocleavage half-lives obey an inverse-linear dependence on light intensity and saturation of the excited state was not found over the range of 5-50 mW/cm² at 365 nm. This indicates that, in principle, even higher intensity light, such as provided by a nitrogen laser, could be used to shorten exposure times.

Solvent effects reveal that photocleavage rates proceeded rapidly under dry conditions, or when the substrate was maintained under a nonpolar solvent such as toluene or dioxane. To date, most of the photoremovable protecting groups have been derivatives of 2-nitrobenzylic compounds. Both the structure of the nitrobenzyl moiety and the atom to which it is attached have some effect on the efficiency and wavelength required for cleavage. By changing substituents in the aromatic ring and at the benzylic carbon, improved efficiency of deprotection can be accomplished. Also, different types of protecting groups that exhibit much higher photolysis rates and quantum yields can be used. One of the possible candidates can be desoxybenzoinyl (desyl) derivatives, which have much higher photolysis quantum rates and therefore can be cleaved much faster. In addition, the photo by-product is inert and photolysis is efficiently performed at 360 nm.

High density arrays of oligonucleotide (or other) probes are an emerging technology for research and potential clinical diagnostics. Arrays of up to 300,000 oligos or more may be manufactured using conventional photolithographic methods. These arrays are used for resequencing and expression studies via hybridization to the array. These chips currently have feature sizes of 20 microns.

The present invention can provide a handling system for the design, deposition and formation of biological samples on slides. Furthermore, unlike biochips that are expensive to make and have a reduced yield due to the underlying electronics, the present invention does not suffer from high initial cost to set up a manufacturing run. Nor does the present invention require a long time to make a sequence change on the array.

One example of slide sample array preparation for use with the micromirror imager disclosed herein is the use of light catalyzed chemistry. Light catalyzed chemistry can be used to attach to, e.g., a glass slide, nucleic and amino acids, lipids, carbohydrates or inorganic or organic molecules that can be used to detect known and unknown molecules. For example, nucleic acids segments, such as oligonucleotides can be attached to detect the presence of complementary or hybridizing nucleic acids. The strength of the interaction between the nucleic acid on the slide and the analyte can be varied as is known to those of skill in the art, e.g., changes in salt concentration, temperature of hybridization, etc. Interactions with proteins and even cells can be measured by attaching, e.g., receptors or ligands to the slide surface to measure binding. As with nucleic acid interactions, interactions with receptors or ligands can be affected by the presence or absence or, e.g., cofactors, competitors and the like.

As an example, the formation of nucleic acids arrays on a substrate surface is used as an example. More conventional chemistries may also be used to attach molecules to the substrate surface, depending on the nature of the substrate, the molecules that are being attached and other factors that will be known to those of skill in the art of chemical attachment and synthesis.

The chemistry for light-directed oligonucleotide synthesis using photo labile protected 2′-deoxynucleoside phosphoramites has been developed at, e.g., Affymetrix, U.S.A. The basics of one type of photo-labile protection chemistry are explained in U.S. Pat. No. 5,424,186, wherein relevant explanations of basic photochemistry techniques and compounds are incorporated herein by reference.

For example, the reaction of commercially available 3,4-(methylenedioxy) acetophenone with nitric acid followed by ketone reduction, and treatment with phosgene gives chloroformate. Then 5′-hydroxyl of N-acyl-2′-deoxynucleosides reacts with chloroformate, and 3′-hydroxyl reacts with 2′cyanoethyl N,N,N′,N′-tetraisopropylphosphorodiamidite to yield photo labile protected phosphoramidites.

Standard phosphoramidite chemistry is adapted to include photo labile protecting groups by replacing the 5′-protecting group DMT, and incorporating photoactivateable hydroxyl linker into the synthesis substrate. Hydroxyl groups are selectively deprotected by irradiation at a wavelength of 365 nm, and oligonucleotides assembled using standard phosphoramidite chemistry.

Probes for the array are chosen to be complementary to regions of the genome at the desired probe frequency (which defines the resolution of the mapping) and over the genomic region of interest (which defines the coverage). For example, selection of probes to be complementary to a sequence every 10,000 base pairs (10 kbp resolution) covering the entire human genome will result in an array of approximately 300,000 probes. The array may comprise probes that have been selected by visual inspection of the sequences to be probed or probes that have been selected by automated computational means. A process for automated selection of oligonucleotide probes is described below. Because the present invention is most advantageous when probing a large number of sites in parallel, the preferred method of probe choice is by automated computational means.

Oligonucleotide arrays of greater than 300,000 probes are accessible with currently available methods of array fabrication. Thus, the entire genome, including non-coding regions, can be analyzed at a resolution better than 10,000 basepair with a single array. This resolution is better than that obtained with arrays of BAC clones and covers more of the genome than an array of cDNA. The present invention also has the advantage over other methods of not requiring the collection of biological material such as clones, chromosomes, or cDNAs in order to make the array, but only requires sequence information.

The array of the present invention is capable of the one or more wavelength detection required expression level analysis of, e.g, at 20, 25, 30, 50, 100, 200, 500, 1,000, 2,000, 20,000, 200,000 or even 2,000,000 or more independent DNA binding elements deposited or created on a substrate, e.g., glass slides. To deposit that many samples of a single slide, a microchemical spotting system may be used. Alternatively, other slide spotting systems may be built using array technologies such a photolithographic techniques and photodeprotection chemistry.

High density arrays of oligonucleotide (or other) probes are an emerging technology for research and potential clinical diagnostics. Arrays of up to 65,000 oligos, manufactured using photolithographic methods are now available commercially from Affymetrix/Hewlett Packard. These arrays are used for resequencing and expression studies via hybridization to the array. These chips currently have feature sizes of 20 micron or less. The present invention provides and alternative handling and data acquisition and analysis system for the analysis of biological sequence on slides as set forth in Appendix A.

The present invention takes advantage of slide spotter systems in conjunction with a hyperspectral reader for gene expression analysis. A slide spotter may be constructed from, e.g., a Toshiba high precision/reproducibility pick and place robot with a multi-channel spotting head. The robot is programmable from a teach pendant or via PC computer. Different types of print heads may be used to spot slides, e.g., a pin spotter, a microvalve/capillary spotter or a piezoelectric/capillary spotter. These provide options of increasing accuracy, complexity and risk. An ultra clean environment is maintained using a HEPA filter to pressurize robot operating volume and proper clean room practices. Microwell plates are kept cool using a surface chiller to minimize evaporation.

Specifications for a slide spotter can include a spot volume of 500 picoliters to 10 nanoliters, a total volume deposited of 500 picoliters (if used with 40 slides this requires 20 nanoliters to 400 nanoliters of volume), and a total sample prime volume of 2 microliters. A drop size for use with slide spotting may be 90 picoliters (e.g., a piezo shooter system, 0.5-1.0 nanoliters for microvalve, or 1-10 nanoliters for pin tool). The system should provide a spot reproducibility of approximately >95%. Shoot times of 6 milliseconds (piezo) to 0.1 seconds (microvalve or pin tool) may be used. Spot dimensions may be of up to about 100 microns on a slide size of, e.g., one inch x three inches. A post grid or orientation may be of 48×144 post, with a slide spot area of 0.75×2.25 inches (about 19 mm×57 mm). The distance between spots may be of about 0.19 mm/48 spots which totals 396 microns. The X-Y step size and reproducibility of a Toshiba robot is about 20.3 microns, which yields an X-Y step between spots of 396 microns/20.3 microns to give 19 spots.

In one example of a slide spotter a 384-well plate may be used, with up to about 18 384-well plates kept on a chilled plate to control evaporation. In this example, the samples “on deck” or queued in the plates may be of about 6,912. Slides on deck may be, e.g., forty, if six potter pins/shooters are used per robot arm. Basic functions or steps per cycle can include: clean, aspirate, prime/verify shooter, and spot.

In operation, the hyperspectral slide reader may be used for, e.g., expression analysis. Polymerase Chain Reaction Polymerase Chain Reaction (PCR) products, cDNAs, oligonucleotides, DNA fragments genomic DNA have been spotted on glass as high-density hybridization targets. Fluorescently labeled cDNAs derived from cellular extracts of mRNA have achieved a dynamic range (detection limit) of 1 in 10,000 to 100,000, allowing for detection of message in low and high abundance. Many studies to measure differential expression have been reported for yeast, insect, animal and human DNAs. Presently, comprehensive and concise data on quantitative analysis of gene expression are available. Use of known expression data may be used to predict and measure known expression patterns having clinical/clinical research application with unknown samples to obtain real-time expression data.

The present invention may be used with existing photochemical protocols and slide spotting technology, in conjunction with known expression levels for preselected and known genes, to optimize gene expression analysis using multiplexing of query samples by using a number of dyes and the full spectral imaging capabilities of the a slide reader of an array. A hyperspectral slide reader, e.g., may be used to identify the expression levels of every gene of the entire organism at one time for multiple multiplexed samples.

In operation, the present invention may be used as follows. A test sample of genomic DNA is obtained (e.g., DNA from diseased tissue), as is a sample of reference DNA (e.g., DNA from normal tissue). Each sample, test and reference, is labeled with a different fluorescent tag. By using more than two dyes, multiple test samples can be compared to a single reference in one study. Additional labels can also be used for an RNA sample in order to profile transcript levels simultaneously. Many methods are known for the incorporation of a fluorescent label into a nucleic acid sample, including but not limited to nick translation and incorporating labeled monomers into PCR products using random primers. Hybridization to oligonucleotide probes is most efficient for DNA fragments shorter than 150 bases, because it requires less disruption of intrastrand base pairing or more facile displacement of complementary strands. Therefore, the preferred sample preparation method not only labels the sample but also fragments it. The protocol included below for labeling of genomic DNA by nick translation results in labeled DNA with a distribution of fragment sizes between 50 and 150 nucleotides.

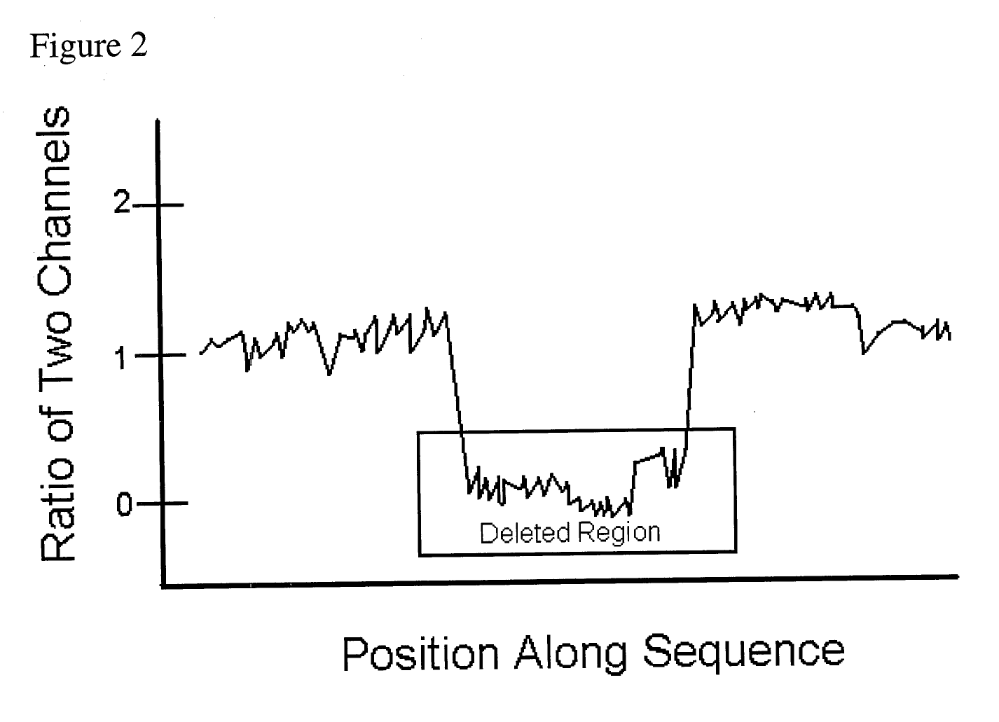

The labeled samples are then mixed and applied to the array of probes. Unlabeled DNA to block non-specific binding of repetitive sequences may also be included. COT DNA may be used for this purpose. The sample DNA is allowed to bind to the array for an appropriate period of time, and the unbound and weakly bound DNA is subsequently washed away. The fluorescence due to each dye is then measured in each feature. The ratio of the signal of the dye in the test sample to the dye in the reference sample is then calculated as a function of the position in the genome. The ratios may be normalized such that they are predominantly centered around 1.0. Features for which the ratio varies singificantly from 1.0 indicate a deletion or amplification in the region those features probe. The result of a deleted region is illustrated schematically in FIG. 2.

The probes of the array need not be restricted to DNA. Any molecule that binds to DNA with sequence specificity can be used. Examples of possible probe molecules include, but are not limited to, RNA, peptides, minor groove-binding polyamides, peptide nucleic acids (PNA), locked nucleic acids (LNA), and 2′-O-methyl nucleic acid.

Comparative Genomic Hybridization (CGH) Probe Design Procedure

The following is a brief protocol that sets-forth the design of the probes for positioning on the array. Also attached as Appendix A is a sample program that implements the system of the present invention, in which the operation of the program serves to design the sequences that will be used on the array to the specific portion of the genome that is to be analyzed. As will be apparent to those of skill in the art, the portion of the genome that may be analyzed using the present invention includes the entire genome, selected portions of the genome (e.g., specific chromosomes). Using the program and the methodology set-forth in the program, the skilled artisan inserts the sequence for which unique oligonucleotides are to be designed and printed on the array, but may be further automated to direct the deposition and creation of the array using automated apparatus and methods.

The resolution of the CGH analysis will be driven by two factors, the number of specific locations on the array (e.g., 20, 30, 50, 100, 200, 1,000, 2000, 5,000, 20,000, 50,000, 100,000, 200,000, 500,000, 1,000,000, 2,000,000 or greater) and the extent of the genome that is to be analyzed by CGH. For example, is a resolution of 10 kB is sought, then the entire genome may be probed on a single array of 300,000 locations. On the other hand, if a resolution of 20 bp is sought, then the artisan may use 30 arrays of 300,000 unique locations to scan, e.g., a human genome. Other genomes, of course, may be scanned and CGH determined using the present invention.

1. User inputs sequence file in 5′ to 3′ direction.

2. The complement sequence from step two is generated in the 3′ to 5′ direction.

3. The parent probe list is created from the sequence in step 2. There are two major ways to do this depending on the length of the sequence. First the probes can be right next to each other. Such that the first probes starts at position X and extends specified number of bases, N. The second probe would start at position X+N, and extend N bases. A second method would put a buffer of sequence, Y, between probes. In this method the first probe would start at X and extend to X+N. The second probe would start at X+N+Y and extend N bases.

4. The parent probe list is filtered to remove probes that are deemed not to be suitable for hybridization analysis. Factors such as low complexity sequence are taken into account.

5. A file is outputted containing all the probes for each position in the reference sequence.

Labeling Protocol

1. In a single PCR tube combine all of the following using NT buffer, nucleotide mix, and NT enzyme mix provided by Promega for nick translation:

40 ul of genomic DNA (8 ug/ 40 ul).

80 ul of Nucleotide mix.

40 ul of NT buffer.

10 ul of Cy 3 dCTP.

40 ul of NT enzyme mix.

190 ul autoclaved water.

400 ul total reaction volume.

2. Divide the 400 ul rxn volume into 8 PCR tubes (˜50 ul per tube).

3. Incubate at 15° C. for 25 hours followed by 4 hrs at 24° C.

4. Immediately add 5 ul of stop solution to each tube while still at 24° C. Then, heat to 65° C. for 15 min. Then maintain at 4° C.

5. Combine all 8 PCR tubes.

6. Phenol extract.

7. De-salt with a NAP-5 column (Pharmacia).

8. Ethanol precipitate.

FIG. 1 is an illustrative drawing showing the signal detected from an array wherein an increasing number of labeled probe is attaching to an array at a specific location. As increasing probes hybridize to the array a stronger signal is detected.

FIG. 2 is a graph showing the comparative genomic hybridization signal detected on an array using the present invention for a deleted locus when comparing the baseline signal to the expected location of a gene. Where a deletion occurs, not only can the deletion be detected using the present invention, but also the specific sequences deleted due to the limit of resolution made available using the apparatus, system and methods disclosed herein.

CGH Comparison Using a DOC Micromirror Array.

Genetic deletions and copy number increases are associated with many diseases. For example, the transcriptional effects of deletion of tumor suppressor genes or their regulatory elements are associated with cancer. Copy number increases of oncogenes similarly contribute to tumorigenesis. Furthermore, a number of developmental abnormalities result from gain or loss of a chromosome or a region of a chromosome. Comparative genomic hybridization (CGH) is a technique that provides a genome-wide map of DNA sequence copy number as a function of location on the chromosome. CGH is typically carried out by co-hybridization of two labeled genomic samples to arrays of large genomic DNA clones (e.g., BACs) or cDNAs. Genomic DNA isolated from two cell lines was labeled in separate nick translation reactions with Cy3 and Cy5. One sample was from the lung tumor cell line HI 299. The other was from the B lymphocyte cell line BL2009.

An oligonucleotide array was made using a digital optical chemistry (DOC) synthesizer (U.S. Pat. No. 6,295,153 by Garner, relevant portions incorporated herein by reference), for CGH. Comparative genomic hybridization was conducted on an 8,000-gene DOC microarray. Oligonucleotide arrays have the advantages of high sequence resolution of chromosomal abnormalities and the ability to probe specifically for abnormalities in non-coding regions. In one such study, a CGH array was generated comprising 86,000; 23nucleotide probes spanning the human genome.

In operation, total genomic DNA from two cell lines was labeled and hybridized to the DOC made chip. The cell lines used in this study were BL2009 (ATCC # CRL-5961), a blood lymphocyte cell line with no reported deletions, and H1299 (ATCC # CRL-5803), a cell line derived from a non-small cell lung carcinoma and containing a partial deletion of the p53 protein. 24 μg of genomic DNA from each cell line was labeled using nick translation (Promega, Wis.) and Cy-labeled dUTP (Amersham, N.J.). The HI 299 DNA was labeled with Cy3 dUTP and the BL2009 DNA with Cy5 dUTP. A hybridization solution was prepared containing approximately 18 μg each of Cy5-labeled BL2009 DNA and Cy3-labeled H1299 DNA together with 3 μg human cot-1 DNA (Invitrogen, Calif.) and 1.6 μM Cy3-labeled control oligonucleotide (a sequence not found in the human genome). This hybridization solution was denatured at 95° C. for 10 minutes.

The samples were then hybridized to the microarray at 37° C. for 18 hours in darkness. The microarray was washed with IL 6×SSPE, 0.01% Tween™ and 250 mls 0.8×SSPE, 0.0017% Tween™ then centrifuged briefly at 3,000 rpm to remove residual 0.8×SSPE, 0.0017% Tween™. Finally, the microarray was scanned using an Applied Precision arrayWoRx e Biochip Reader with exposure settings of 1.0 second in the Cy3 channel and 0.3 seconds in the Cy5 channel. The image files generated by the scanner software were converted to 16-bit TIFF format and analyzed using, e.g., the DOC_U_MENTOR code (UT Southwestern).

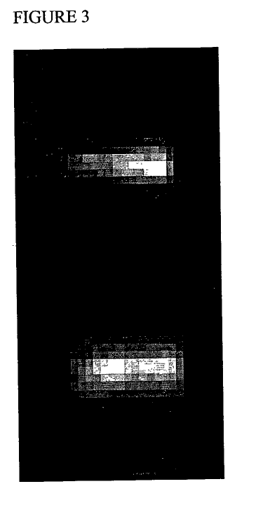

The two samples were co-hybridized on the DOC CGH array and the resulting two-color hybridization signals were quantified. FIG. 3 is an extreme zoomed image of the array showing a small portion of the array. FIG. 3 demonstrates that two bright sections were found, indicating hybridization of the genomic DNA without a need for amplification of the underlying DNA. Each bright section corresponds to 4 distinct probes arranged horizontally, and each probe is a 23 mer oligonucleotide. The data show that a resolution of at least 23 bases is obtained. The DOC microarray and scanning system allows for the customization of an array for conducting ultra-high resolution CGH for any genome. The method, system and analysis of the present invention demonstrate that the detection is customizable, rapid and may be optimized for probes to localize any position in the genome.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method for mapping a sample at high resolution comprising the steps of:
selecting a portion of a genome for genetic analysis;
providing an array of oligonucleotides specific to the portion of the genome selected for genetic analysis; and
conducting a comparative genomic hybridization analysis, whereby the resolution of the analysis is between about 20 and 2,000,000 basepairs.

2. The method of claim 1, wherein the step of conducting a comparative genomic hybridization analysis comprises the steps of:
hybridizing a sample nucleic acid and a reference nucleic acid to the array; and
detecting the binding of the sample nucleic acid to specific locations on the array that contain specific oligonucleotides specific for portions of the genome selected for genetic analysis.

3. The method of claim 1, further comprising the step of creating a map of the portion of the genome detected.

4. The method of claim 3, wherein the limit of resolution of the genetic map is between about 20 bases and 200 kilobases.

5. The method of claim 3, wherein the limit of resolution of the genetic map is between about 200 bases and 100 kilobases.

6. The method of claim 3, wherein the limit of resolution of the genetic map is between about 5 and 50 kilobases.

7. The method of claim 3, wherein the limit of resolution of the genetic map is between about 10 and 15 kilobases.

8. The method of claim 1, wherein the sample comprises a histological sample.

9. The method of claim 1, wherein the sample comprises a blood sample.

10. The method of claim 1, wherein the sample nucleic acid comprises DNA.

11. The method of claim 1, wherein the sample nucleic acid comprises genomic DNA.

12. The method of claim 1, wherein the sample nucleic acid comprises mRNA.

13. The method of claim 1, wherein the sample comprises a fluorescent, enzymatic, radiolabelled or physical marker.

14. The method of claim 1, wherein the reference comprises a fluorescent, enzymatic, radiolabelled or physical marker.

15. The method of claim 1, wherein the array is further defined as comprising a microarray of nucleic acid oligomers on a slide.

16. The method of claim 1, wherein the array is further defined as comprising a microarray of nucleic acid oligomers on a wafer.

17. A method for high-throughput high resolution mapping of a sample comprising the steps of:
selecting a portion of a genome for genetic analysis;
providing an array of oligonucleotides specific to the portion of the genome selected for genetic analysis;
hybridizing a sample nucleic acid and a reference nucleic acids to the array;
detecting the binding of DNA to specific portions of the genome; and
conducting a comparative hybridization analysis between the sample and the reference nucleic acids.

18. The method of claim 17, further comprising the step of creating a map of the portion of the genome detected.

19. The method of claim 17, wherein the limit of resolution of the genetic map is between about 20 bases and 200 kilobases.

20. The method of claim 17, wherein the limit of resolution of the genetic map is between about 200 bases and 100 kilobases.

21. The method of claim 17, wherein the limit of resolution of the genetic map is between about 5 and 50 kilobases.

22. The method of claim 17, wherein the limit of resolution of the genetic map is between about 10 and 15 kilobases.

23. The method of claim 17, wherein the sample comprises a histological sample.

24. The method of claim 17, wherein the sample comprises a blood sample.

25. The method of claim 17, wherein the sample nucleic acid comprises DNA.

26. The method of claim 17, wherein the sample nucleic acid comprises genomic DNA.

27. The method of claim 17, wherein the sample nucleic acid comprises mRNA.

28. The method of claim 17, wherein the sample comprises a fluorescent, enzymatic, radiolabelled or physical marker.

29. The method of claim 17, wherein the reference comprises a fluorescent, enzymatic, radiolabelled or physical marker.

30. The method of claim 17, wherein the array is further defined as comprising a microarray of nucleic acid oligomers on a slide.

31. The method of claim 17, wherein the array is further defined as comprising a microarray of nucleic acid oligomers on a wafer.

32. The method of claim 17, wherein in which two different wavelengths are used stain and thereby detect the sample and the reference nucleic acids.

33. The method of claim 17, wherein the reference is genomic DNA and the sample is RNA, wherein the difference in signal is used to detect the expression of one or more genes.

34. The method of claim 17, wherein the reference is genomic DNA and the sample is RNA, wherein the difference in signal is used to detect the expression of one or more genes that are undergo alternative splicing.

35. An apparatus for comparative genomic mapping of a sample and a reference, comprising:
an array of oligonucleotides having a surface derived from a selected portion of a genome;
a reader capable of producing a signal disposed to detect events that occur at the surface of the array of oligonucleotides; and
a computer attached to the reader, wherein the computer processes a signal from the reader to determine the relative intensity produced at the array surface and capable of comparing the intensity of the signal from the array to provide map with a resolution between 20 basepairs and 2,000,000 basepairs.

36. The apparatus of claim 35, wherein the limit of resolution of the genetic map is between about 20, 30, 50, 100, 200, 1,000, 2000, 5,000, 20,000, 50,000, 100,000, 200,000, 500,000, 1,000,000, and 2,000,000 base pairs.

37. The apparatus of claim 35, wherein the limit of resolution of the genetic map is between about 200 bases and 100 kilobases.

38. The apparatus of claim 35, wherein the limit of resolution of the genetic map is between about 5 and 50 kilobases.

39. The apparatus of claim 35, wherein the limit of resolution of the genetic map is between about 10 and 15 kilobases.

40. The apparatus of claim 35, wherein the sample comprises a histological sample.

41. The apparatus of claim 35, wherein the sample comprises a blood sample.

42. The apparatus of claim 35, wherein the sample comprises DNA.

43. The apparatus of claim 35, wherein the sample comprises genomic DNA.

44. The apparatus of claim 35, wherein the sample nucleic acid comprises mRNA.

45. The apparatus of claim 35, wherein the sample comprises a fluorescent, enzymatic, radiolabelled or physical marker.

46. The apparatus of claim 35, wherein the reference comprises a fluorescent, enzymatic, radiolabelled or physical marker.

47. The apparatus of claim 35, wherein the array is further defined as comprising a microarray of nucleic acid oligomers on a slide.

48. The apparatus of claim 35, wherein the array is further defined as comprising a microarray of nucleic acid oligomers on a wafer.

49. The apparatus of claim 35, wherein in which two different wavelengths are used stain and thereby detect the sample and the reference nucleic acids.

50. The apparatus of claim 35, wherein the reference is genomic DNA and the sample is RNA, wherein the difference in signal is used to detect the expression of one or more genes.

51. The apparatus of claim 35, wherein the reference is genomic DNA and the sample is RNA, wherein the difference in signal is used to detect the expression of one or more genes that are undergo alternative splicing.

52. A system for oligonucleotide design comprising the steps of:
inputting a portion of a genome sequence file in 5′ to 3′ direction;
generating a complementary sequence in the 3′ to 5′ direction to create a parent probe list; and
creating a final probe list by:
filtering probe sequences from the final probe list that are not suitable for hybridization analysis from the parent probe list; and
outputting a file containing all the probes for each position in the reference sequence.

53. The system of claim 52, wherein the step of generating probes creates probes from the final probe list with adjacent non-overlapping sequences.

54. The system of claim 52, wherein the step of generating probes creates probes from the final probe list with adjacent and overlapping sequences.

55. The system of claim 52, wherein the step of wherein the first probe from the final probe list starts at position X and extends specified number of bases, N.

56. The system of claim 52, wherein the step of generating probes from the final probe list generates a second probe that starts at position X+N, and extends N bases.

57. The system of claim 52, wherein the step of generating probes from the final probe list further includes the step of adding a buffer of sequence, Y, between probes.

58. The system of claim 57, wherein a first probe in the final probe list starts at X and extend to X+N and the second probe starts at X+N+Y and extends N bases.

59. The system of claim 57, wherein the step of filtering probes sequences from the final probe list is further defined as removing probe sequences having low complexity from the final probe list.
